(19) United States
(12) Patent Application Publication
(10) Pub. No.: US 2016/0120925 A1
(43) Pub. Date: May 5, 2016

(54) DRUG CONTAINING THYME AND PRIMROSE OR THYME AND IVY AS A PDE-4 INHIBITOR

(71) Applicant: BIONORICA SE, Neumarkt (DE)

(72) Inventor: MICHAEL POPP, Lauf-Heuchling (DE)

(21) Appl. No.: 14/896,186

(22) PCT Filed: Jun. 6, 2014
(86) PCT No.: PCT/EP2014/061923

§ 371 Date: Dec. 4, 2015

(30) Foreign Application Priority Data
Jun. 6, 2013 (EP) 13170956.0

(51) Int. Cl. A61K 36/53; A61K 9/00; A61K 36/185

(52) U.S. Cl. CPC A61K 36/53; A61K 36/185; A61K 9/0014

(57) ABSTRACT

The invention relates to an extract from thyme (Thymus L.) in combination with primrose (Primula veris) or ivy (Hedera helix) for use as PDE-4 inhibitor in the prophylaxis and treatment of selected diseases and to a corresponding pharmaceutical and use thereof.

The invention relates to an extract from thyme (Thymus L.) in combination with primrose (Primula veris) or ivy (Hedera helix) for use as PDE-4 inhibitor in the prophylaxis and treatment of selected diseases and to a corresponding pharmaceutical and use thereof.

Bronchipret® (registered trademark of BIONORICA SE) is a known highly potent pharmaceutical—a phytopharmaceutical, which contains thyme in combination with primrose or ivy. Bronchipret® is preferably distributed in the form of (film-coated) tablets (thyme/primrose—dry extract) and juice (thyme/ivy). The production of the pharmaceutical is known. The secretolytic, bronchospasmolytic, antitussive, antiphlogistic, antibacterial and antiviral effect has been described. The suitability of Bronchipret® for the treatment of COPD and bronchitis has also been described in WO2011/147992 in the name of the applicant.

Moreover, R. van den Hoeven (R. van den Hoeven at al, Study of the effect of the lung function of five Austrian saddle horses suffering recurrent airways obstruction (heaves). The Veterinary Record (2003), 152, 555-557) describes the influence of Bronchipret® on lung function in horses.

Irwin Ziment for example also describes the fact that. Bronchipret® is especially effective for treating bronchitis (Irwin Ziment, Complementary and alternative medicine therapies for chronic obstructive pulmonary disease Focus Altern Complement Ther 2003; 8: 385-91).

The suitability of Bronchipret® for the treatment of acute bronchitis is described in:

Kemmerich B, Eberhardt H, Stammer H. Efficacy and tolerability of a fluid extract combination of thyme herb and ivy leaves and matched placebo in adults suffering from acute bronchitis with productive cough. A prospective, double-blind, placebo-controlled clinical trial. Drug Research 2006; 56(9):652-660; Kemmerich B: Evaluation of efficacy and tolerability of a fixed combination of dry extracts of thyme herb and primrose root in adults suffering from acute bronchitis with productive cough. A prospective, double-blind, placebo-controlled multicenter clinical trial. Drug Research 2007; 57(9):607-615; Marzian O. Treatment of acute bronchitis in children and adolescents. Non-interventional postmarketing surveillance study confirms the benefit and safety of a syrup made of extracts from thyme and ivy leaves [Article in German] MMW Fortschr Med. 2007; 149(11):69-74; Ismail C, Willer G, Steindl H: Bronchipret in cases of acute bronchitis. Schweizerische Zeitschrift für Ganzheitsmedizin (Swiss Journal for Holistic Medicine), 2003; 15: 171-5.

However, there is a high need to provide novel, effective pharmaceuticals for the treatment and prophylaxis of further diseases of the airways and lungs as well as autoimmune diseases.

Surprisingly, Bronchipret®, and more particularly a pharmaceutical composition consisting of thyme and ivy, or thyme and primrose, demonstrates an inhibitory effect on the target enzyme “PDE-4” in the lung of a mammal, in particular of a human (Uzunov, P. and Weiss, B.: Separation of multiple molecular forms of cyclic adenosine 3′,5′-monophosphate phosphodiesterase in rat cerebellum by polyacrylamide gel electrophoresis. Biochim. Biophys. Acta 284:220-226, 1972). Bronchipret® is therefore suitable as a phosphodiesterase-4 inhibitor (“PDE-4 inhibitor”),

There are 11 different phosphodiesterase types (PDE1 to PDE11). Of the PDE4 subtypes, 4 are known (PDE4A to PDE4D), Phosphodiesterase-4 inhibitors (also phosphodiesterase IV inhibitors) are substances that specifically inhibit the enzyme phosphodiesterase IV. Phosphodiesterase IV causes a conversion and breakdown of the messenger substance cAMP (cyclic adenosine monophosphate), which has an anti-inflammatory effect, into AMP. The inflammation-promoting substances thus dominate. PDE-4 inhibitors, however, advantageously increase the concentration of intracellular cAMP. Cyclic AMP (cAMP) plays a key role in the overcoming of inflammatory reactions. An increased lifetime of cAMP conveyed by PDE-4 inhibitors causes an anti-inflammatory effect.

A pharmaceutical not permitted since 1979 as an archetype of a phosphodiesterase-4 inhibitor is the active substance rolipram, which was developed for the treatment of depression and multiple sclerosis. PDE-4 inhibitors act in any case in an anti-inflammatory manner and were tested inter alia in the fields of application constituted by COPD, bronchial asthma, depression and multiple sclerosis. Specific inhibitors of isoform 4 (PDE4) were used for example in the treatment of COPD (for example Daxas®). Previously, only one active substance was permitted as pharmaceutical for the treatment of COED, more specifically roflumilast (Daxas®). The development of cipamfylline and other PDE4 inhibitors was discontinued, but others are currently being developed. (for example apremilast).

Bronchipret® has thus proven to have a curative and palliative effect for the prophylaxis and treatment of diseases of the airways and lungs and also of autoimmune diseases selected from the group of diseases linked by PDE-4 inhibitors.

The invention therefore relates to a pharmaceutical containing thyme and ivy or thyme and primrose, in particular an extract from thyme and ivy or thyme and primrose, for use or application in the prophylaxis and treatment of diseases of the airways and lungs and of autoimmune diseases selected from the group of diseases linked by PDE-4 inhibitors, more specifically:

Airways and Lungs:

Diseases of the asthmatic type, selected from the group consisting of atopic asthma, non-atopic asthma, allergic asthma, atopic bronchial IgE-mediated asthma, bronchial asthma, essential asthma, intrinsic asthma caused by pathophysiologic disturbances, extrinsic asthma caused by environmental factors, essential asthma of unknown or unapparent cause, bronchitic asthma, emphysematous asthma, exercise-induced asthma, allergen induced asthma, allergic asthma, cold air induced asthma, occupational asthma, infective asthma caused by bacterial, fungal, protozoal, or viral infection, non-allergic asthma, incipient asthma and wheezy infant syndrome,

bronchiolitis, chronic or acute bronchoconstriction, small airways obstruction, and emphysema; obstructive or inflammatory airways diseases of whatever type, etiology, or pathogenesis, in particular an obstructive or inflammatory airways disease that is a member selected from the group consisting of chronic eosinophilic pneumonia, adult respiratory distress syndrome (ARDS),

exacerbation of airways hyper-reactivity consequent to other drug therapy and airways disease that is associated with pulmonary hypertension,

Diseases included in the category of bronchitis, selected from the group consisting of acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,

acute lung injury, emphysema, post-traumatic pulmonary insufficiency,

postinfectious cough (also: subacute cough),

bronchiectasis of whatever type, etiology, or pathogenesis, in particular diseases included in the category of bronchiectasis selected from the group consisting of cylindric bronchiectasis, sacculated bronchiectasis, fusiform bronchiectasis, capillary bronchiectasis, cystic bronchiectasis, dry bronchiectasis and follicular bronchiectasis.

Autoimmune Diseases:

Autoimmune diseases of the lung and skin selected from the group consisting of systemic lupus erythematosus, polyarteritis nodosa, Goodpasture's syndrome, scleroderma, wegner's granulomatosis, sjögren's syndrome, hamman-rich syndrome, sarcoidosis, dermatitis, dermatitis herpetiformis duhring, atopic dermatitis, psoriasis, dermatomyositis.

Within the scope of this invention, diseases of the airways and lungs or autoimmune diseases selected from the group of diseases linked by PDE-4 inhibitors means diseases as specified above. The specified indications and causes and etiology thereof can be deduced for example from Mutschler, E. et al., Arzneimittelwirkungen (Pharmaceutical Effects), 8th edition, wiss. Verl. Ges. mbH, Stuttgart, 2001 or Pschyrembel®, de Gruyter, Berlin, 2012. In addition, said indications are recorded in TCD-10-WHO Version 2013, International statistical Classification of Diseases and related Health Problems, 10th Revision Version 2013 (online). The aforementioned indication “postinfectious cough” outlasts the triggering bronchial infection by a number of weeks. After some acute virus infections it may be eight weeks until the postinfectious cough subsides spontaneously (Kardos P at al. Leitlinie der Deutschen Gesellschaft für Pneumologie und Beatrmungsmedizin zur Diagnostik und Therapie, Pneumologie (Guideline of the German Society for Pneumology and Respiratory Medicine for Diagnostics and Therapy, Pneumology) 2010; 64: 336-373).

The invention also relates to the use or application of thyme and ivy or thyme and primrose or a respective extract thereof for producing a pharmaceutical for the prophylaxis and treatment of diseases of the airways and lungs and also of autoimmune diseases selected from the group of PDE-4-linked diseases.

In accordance with the invention it is also advantageous that thyme and ivy or thyme and primrose or a respective extract thereof allow an increase of the mucocillary clearance. Mucocillary clearance is a mechanism in which both foreign particles and mucus from bronchi and the windpipe are transported away upwardly. For this purpose, the epithelial cells are provided with cilia, which beat rhythmically and move the mucus along. A restricted beat frequency (CBF) leads to a weakening of the transporting away of the mucus and is compensated for by coughing. Driving the beat frequency can therefore remedy coughing and reduce the mucus-conveyed obstruction of the airways. Surprisingly, thyme and ivy or thyme and primrose or a respective extract thereof demonstrate an advantageous induction of the ciliary beat frequency, such that the curative and palliative effect of the diseases linked by PDE-4 inhibitors is positively assisted.

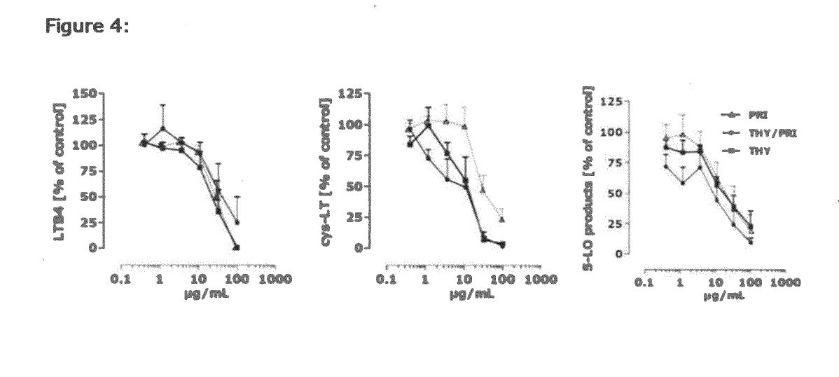

In addition, thyme and ivy or thyme and primrose or a respective extract thereof demonstrate an advantageous secondary effect of protection against inflammation, as evidenced in Example 4 and FIG. 4, which is provided in addition to PDE-4 inhibition and substantiates a supporting rationale for the aforementioned indications according to the invention.

The specified plant (drugs) according to the invention for the pharmaceutical according to the invention can be obtained, as is conventional for the respective plant drug, from preferred plant parts, such as the leaf, root, etc., as described correspondingly (for example see Ph. Eur., European Pharmacopoeia). In particular, aqueous and/or ethanolic extracts can be used or dry extracts obtainable therefrom. Reference is made to the technical teaching forming the subject matter of EP 136860531 and EP 075330631 with regard to the production of the extracts according to the invention.

The pharmaceuticals of the present invention can be produced advantageously and in the manner known per se and can be used in the form of a galenic formulation corresponding to their application.

The galenic formulations of the pharmaceuticals according to the invention are characterised in that they include oral formulations such as dragées, tablets, film-coated tablets, powder, capsules or liquid dilutions, in particular drops, juices or syrups.

In the case of topical application or use, sprays, ointments, emulsions, powders, grains, liquid or solid preparations for inhalation, compresses, tamponades, tonsil brush solutions or gargling solutions are suitable in particular.

The invention also relates to a pharmaceutical formulation containing a pharmaceutical according to the invention. The pharmaceuticals according to the invention can be prepared in the form of pharmaceutical preparations or compositions in metred units. This means that the preparation or composition is present in the form of individual parts, for example tablets, dragées, capsules, pills, suppositories and ampoules, of which the active substance content corresponds to a fraction or a multiple of an individual dose. The metred units may contain, for example, 1, 2, 3 or 4 individual doses or ½, ⅓ or ¼ of an individual dose. An individual dose preferably contains the quantity of active substance which is administered with an application and which usually corresponds to an entire daily dose, half a daily dose, a third of a daily dose or a quarter of a daily dose.

The term non-toxic, inert pharmaceutically suitable carrier substances is to be understood to mean solid, semi-solid or liquid diluents, fillers and formulation aids of any type.

The term pharmaceutical also includes agents of identical suitability, such as food supplements or even foodstuffs, provided the advantageous effect according to the invention of the agent is implemented.

Preferred pharmaceutical formulations are tablets, dragées, capsules, pills, granulates, suppositories, solutions, juice, suspensions and emulsions, pastes, ointments, gels, creams, lotions, dusts and (nasal) sprays. Tablets, dragées, capsules, pills and granulates may contain the active substance (s) in addition to the conventional carrier materials, such as a) fillers and extenders, for example starches, lactose, sucrose, glucose, mannitol and silica, b) binders, for example carboxymethyl cellulose, alginates, gelatins, polyvinyl pyrrolidone, c) humectants, for example glycerol, d) exploders, for example agar-agar, calcium carbonate and sodium carbonate, e) solution retarders, for example paraffin, and f) resorption accelerators, for example quaternary ammonium compounds, g) wetting agents, for example cetyl alcohol, glycerol monostearate, h) adsorption agents, for example kaolin and bentonite, and i) lubricants, for example talcum, calcium stearate and magnesium stearate, and solid polyethylene glycols, or mixtures of the substances specified under a) to i).

The tablets, dragées, capsules, pills, and granulates may be provided with the usual coatings and casings, which may or may not contain opacifying agents, and may also be composed such that they only or preferably only deliver the active substances in a certain part of the intestinal tract in a delayed manner as necessary, wherein polymer substances and wax for example can be used as embedding compounds. The active substance(s) may also be present in microencapsulated form, where necessary with one or more of the above-mentioned carrier substances.

Suppositories, besides the active substance(s), may also contain conventional water-soluble or water-insoluble carrier substances, for example polyethylene glycols, fats, for example cocoa fat and higher esters (for example C14 alcohol with C16 fatty acid) or mixtures of these substances).

Ointments, pastes, creams and gels, besides the active substance(s), may also contain the conventional carrier substances, for example animal and plant fats, waxes, paraffins, starch, astragalus cellulose derivatives, polyethylene glycols, silicones, bentonites, silica, talcum and zinc oxide or mixtures of these substances.

Powders and sprays, besides the active substance(s), may also contain the conventional carrier substances, for example lactose, talcum, silica, aluminium hydroxide, calcium silicate and polyamide powder or mixtures of these substances. Sprays may additionally contain the conventional propellants. Solutions and emulsions, besides the active substance(s), may also contain the conventional carrier substances, such as solvents, solubilising agents, and emulsifiers, for example water, ethyl alcohol, isopropyl alcohol, ethyl carbonate, ethyl acetate, benzyl alcohol, benzyl benzoate, propylene glycol, 1,3-butylene glycol, dimethylformamide, oils, in particular cotton seed oil, groundnut oil, corn oil, olive oil, castor oil, and sesame oil, glycerol, glycerol formal, tetrahydrofurfuryl alcohol, polyethylene glycols and fatty acid esters of sorbitan, or mixtures of these substances.

Suspensions, besides the active substance (s), may also contain the conventional carrier substances, such as liquid diluents, for example water, ethyl alcohol, propylene glycol, suspension agents, for example ethoxylated isostearyl alcohols, polyoxyethylene sorbitan esters and sorbitan esters, microcrystalline cellulose, aluminium metahydroxide, bentonite, agar-agar, and astragalus, or mixtures of these substances. The specified formulation forms may also contain dyes, preserving agents, and smell- or taste-enhancing additives, for example peppermint oil and eucalyptus oil and sweeteners, for example saccharin.

Further advantages and features of the present invention will emerge on the basis of the description of exemplary embodiments. The following examples and figures serve to explain the invention, without limiting the invention to these examples.
1. Inhibition of the Phosphodiesterase-4 activity

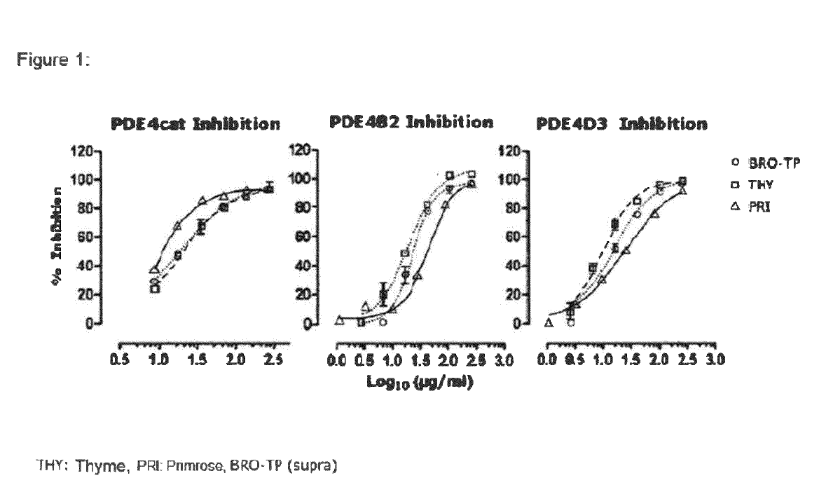

In an in vitro standard experiment it could be shown that the mixture of thyme and primrose dry extract (“BRO-TP”) is able to inhibit the activity of the catalytic domain of the PDE4 enzyme in a concentration-dependent manner. IC50 was 21-26 μg/mL. The individual extracts likewise inhibited the activity of the catalytic domain (thyme: approximately 20 μg/mL; primrose: 10-12 μg/mL). In the further experiments the specific effect on the PDE4 subtypes PDE4B and PDE4D was examined. Here too, inhibitory activity could be demonstrated for all extracts: in the case of PDE4B2, the IC50 values of BRO-TP, thyme and primrose were 26, 22 and 51 μg/mL, and for PDE4D3 were 17, 11 and 28 μg/mL (see FIG. 1).

Methodology: The catalytic domain (PDE4Cat) and the enzyme subtypes PDE4B2 and −D3 were incubated with 6 concentrations of BRO-TP (3.07-300 μg/mL), and the catalytic activity was determined in a radiometric assay. The active substance rolipram (supra) was used as positive control.
2. Induction of Ciliary Beat Frequency (OFF)

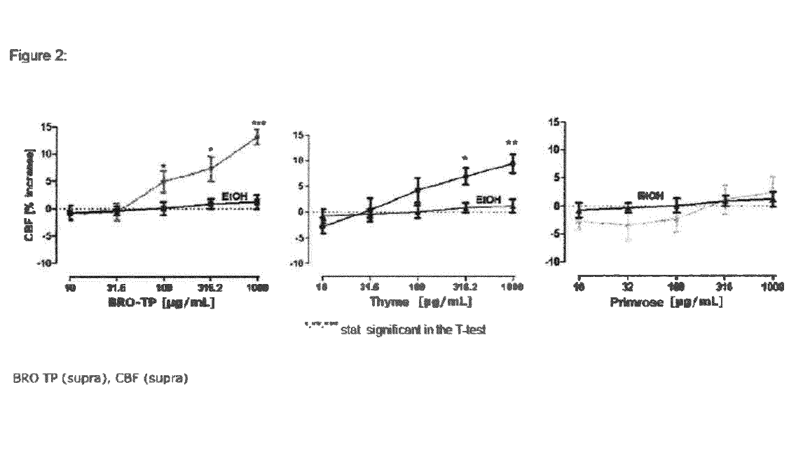

In an ex vivo experiment thin lung slices (Precision-Cut Lung Slices—PCLS) were treated with the thyme/primrose extract mixture, and the individual extracts and the beat frequency of the epithelial cilia were analysed. The treatment with BRO-TP (supra) led from 100 μg/mL to a considerable rise in CBF compared with thyme or primrose alone (see FIG. 2).

Methodology: PCLS 300 μm thick were obtained from guinea pig lungs and transferred into 24-well plates with medium. Rising concentrations of the extracts were added to the medium (10 μg/mL, 32 μg/mL, 100 μg/mL, 320 μg/mL, and 1000 μg/mL) and the beat frequency at the ciliary edge of the airways was analysed by video microscope. By way of comparison, the influence of the vehicle (0.005-0.5% ethanol) was determined. The maximum frequency rise was determined by addition of methacholine (10 μM).

It could be shown that the anti-inflammatory effect of the thyme/primrose dry extract mixture is conveyed inter alia via an inhibition of the PDFA enzyme activity. In addition, an ex vivo cilial beat-stimulating effect is produced by the thyme extract and can be caused by the extract mixture. The results were ascertained in models that are relevant for the pathogenesis and therapy of the claimed indications and therefore provide sufficient proof that in particular the combination of thyme and primrose (dry) extract, as contained in Bronchipret®, is also effective in the treatment of patients.
3. BRO-TP TNFalpha Release in vitro (Anti-Inflammation)

Model: TNFalpha (tumour necrosis factor alpha) release from LPS (lipopolysaccharide)-stimulated immune cells

Protocol—LPS (dexamethasone): Human PBMO in 96-well plates in culture medium (RPMI 1640, 10% PBS, 1% Pen/Strep, 2 mM alanyl-L-glutamine), cell density: 5E⁴ cells/well (cytokine plate), 2E⁵ cells/well (alamarBlue plate). Incubation of the cells for 1 h at 37° C. in 5% CO₂, addition of the components and dexamethasone (vehicle control=0.15% EtOH), incubation of the plates for 1 h at 37° C. in 5% CO₂, subsequent addition of LPS and incubation of the plates for 24 h at 37° C. in 5% CO₂, centrifugation (1,000 rpm, 10 min.), collection of the supernatants and freezing at −80° C.

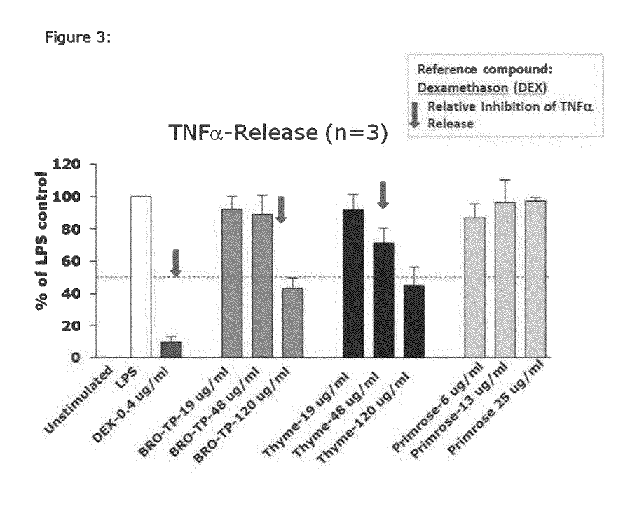

The samples were assessed and the data is presented in FIG. 3.

Summary: BRO-TP inhibits the release of the pro-inflammatory cytokine TNFa from LPS-stimulated human immune cells
4. Leukotriene formation and 5-LO activity

Freshly isolated human neutrophils (5×10⁶ cells/ml for LTB₄ formation) or monocytes (1×10⁶ cells/ml for cys-LT formation) were incubated in PGC buffer for 10 min at 37° C. (with vehicle (0.025% EtOH)), THY/PRI, THY, PRI (0.4-100 μg/mL in each case) or zileuton (3 μM) and then stimulated for 10 min at 37° C. with ionophore A23187 (2.5 μM). The reaction was stopped with ice, and suitable reagents for LTB₄ analysis or for cys-LT analysis were added. LTB₄ was analysed using HPLC, Cys-LTs was analysed using ELISA. The results are presented in FIG. 4.

5-LO was expressed in transformed E. coli B121 and purified using ATP affinity chromatography. In order to determine the 5-LO activity, enzymes were added in a reaction mixture to 5-LO. Samples were incubated for 10 min at 4° C. (with vehicle (0.25% EtOH)), and THY/PRI, THY, PRI (0.4-100 μg/ml in each case), or zileuton (3 μM), preheated at 37° C., and CaCl₂ and arachidonic acid (20 μM) were added. The reaction was stopped after 10 min at 37° C., and prostaglandin B1 (internal standard) was added. Obtained metabolites were analysed using HPLC, more specifically 5-LO products inc. LTB₄ all-trans isomers and 5-H(P)ETE. The results are presented in FIG. 4.

Summary: BRO-TP demonstrates improved anti-inflammatory effects (5-LO) compared with thyme and primrose alone.

1-7. (canceled)

8. A pharmaceutical containing thyme and ivy or thyme and primrose.

9. The pharmaceutical as claimed in claim 8 used as a PDE-4 inhibitor in the prophylaxis and treatment of diseases selected from the group of diseases of the asthmatic type.

10. The pharmaceutical as claimed in claim 8 used as a PDE-4 inhibitor in the prophylaxis and treatment of diseases selected from the group consisting of atopic asthma, non-atopic asthma, allergic asthma, atopic bronchial IgE-mediated asthma, bronchial asthma, essential asthma, intrinsic asthma caused by pathophysiologic disturbances, extrinsic asthma caused by environmental factors, essential asthma of unknown or unapparent cause, bronchitic asthma, emphysematous asthma, exercise-induced asthma, allergen induced asthma, allergic asthma, cold air induced asthma, occupational asthma, infective asthma caused by bacterial, fungal, protozoal, or viral infection, non-allergic asthma, incipient asthma, and wheezy infant syndrome.

11. The pharmaceutical as claimed in claim 8 used as a PDE-4 inhibitor in the prophylaxis and treatment of diseases selected from the group consisting of bronchiolytis, chronic or acute bronchoconstriction, small airways obstruction, and emphysema, obstructive or inflammatory airways diseases of whatever type, etiology, and pathogenesis.

12. The pharmaceutical as claimed in claim 8 used as a PDE-4 inhibitor in the prophylaxis and treatment of diseases selected from the group consisting of an obstructive or inflammatory airways disease that is a member selected from the group consisting of chronic eosinophilic pneumonia, adult respiratory distress syndrome (ARDS), exacerbation of airways hyper-reactivity consequent to other drug therapy and airways disease that is associated with pulmonary hypertension.

13. The pharmaceutical as claimed in claim 8 used as a PDE-4 inhibitor in the prophylaxis and treatment of diseases selected from the group consisting of diseases of acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis, acute lung injury, emphysema, post-traumatic pulmonary insufficiency postinfectious cough; etiology, and pathogenesis.

14. The pharmaceutical as claimed in claim 8 used as a PDE-4 inhibitor in the prophylaxis and treatment of diseases selected from the group consisting of diseases included in the category of bronchiectasis selected from the group consisting of cylindric bronchiectasis, sacculated bronchiectasis, fusiform bronchiectasis, capillary bronchiectasis, cystic bronchiectasis, dry bronchiectasis and follicular bronchiectasis.

15. The pharmaceutical as claimed in claim 8 used as a PDE-4 inhibitor in the prophylaxis and treatment of diseases selected from the group consisting of autoimmune diseases of the lung and skin selected from the group consisting of systemic lupus erythematosus, polyarteritis nodosa, Goodpasture's syndrome, scleroderma, wegner's granulomatosis, sjögren's syndrome, hamman-rich syndrome, sarcoidosis, dermatitis, dermatitis herpetiformis duhring, atopic dermatitis, psoriasis, and dermatomyositis.

16. The pharmaceutical according to claim 8, containing extracts from thyme and ivy or extracts from thyme and primrose.

17. The pharmaceutical according to claim 8, wherein is present in a galenic formulation, oral formulations, tablets, film-coated tablets, capsules, liquid dilutions, a formulation for topical application, solid preparations for inhalation, compresses, tamponades, tonsil brush solutions, gargling solutions or rinsing solutions.

18. The pharmaceutical according to claim 8, characterised in that it is present in a galenic formulation, in dragées, tablets, film-coated tablets, capsules, drops, juices, syrups, as sprays, ointments, emulsions, powders, grains, liquid or solid preparations for inhalation, compresses, tamponades, tonsil brush solutions, gargling solutions or rinsing solutions.

19. The pharmaceutical according to claim 8, wherein the pharmaceutical is present in a galenic formulation.

20. The pharmaceutical according to claim 8, wherein the pharmaceutical is present in an oral formulation such as dragées, tablets, film-coated tablets, capsules, liquid dilutions, in particular drops, juices, syrups or a formulation for topical application.

21. The pharmaceutical according to claim 8, wherein the pharmaceutical is present in sprays, ointments, emulsions, powders, grains, liquid or solid preparations for inhalation, compresses, tamponades, tonsil brush solutions, gargling solutions or rinsing solutions.

22. A pharmaceutical composition containing the pharmaceutical according to claim 8.

23. The pharmaceutical according to claim 8 for topical use.

24. A PDE-4 inhibitor containing thyme and ivy or thyme and primrose or extracts thereof.
